(19) United States
(12) Patent Application Publication
(10) Pub. No.: US 2024/0016159 A1
(43) Pub. Date: Jan. 18, 2024

(54) AGRICULTURAL COMPOSITIONS AND METHODS

(71) Applicant: Carus Animal Health Limited, Reigate (GB)

(72) Inventors: Gregory James Firth, Sheffield (GB); Rajesh Muru Odedra, Stevenage (GB)

(21) Appl. No.: 18/352,676

(22) Filed: Jul. 14, 2023

(30) Foreign Application Priority Data
Jul. 15, 2022 (GB) 2210435.0

(51) Int. Cl. A01N 63/40; A01M 1/02; A01P 1/00; A01N 25/34

(52) U.S. Cl. CPC A01N 63/40; A01M 1/02; A01P 1/00; A01N 25/34; A01M 2200/012

(57) ABSTRACT

The present disclosure relates to compositions and kits for the treatment or prevention of a bacterial disease in a terrestrial plant or animal, comprising a phage and/or an immunogenic agent and a flying insect attractant. The disclosure also relates to methods for attracting flying insects to phages and/or immunogenic agents. The disclosure also relates to the use of a flying insect in a method of transferring a phage and/or immunogenic agent to an animal or plant, and to methods of treating or preventing disease in animals and plants using flying insects. The disclosure also relates to formulations of phages and/or immunogenic agents for use in treating and/or preventing disease in animals or plants.

### FIELD

The present disclosure relates to compositions and kits for the treatment or prevention of a bacterial disease in a terrestrial plant or animal, comprising a phage and/or an immunogenic agent and a flying insect attractant. The disclosure also relates to methods for attracting flying insects to phages and/or immunogenic agents. The disclosure also relates to the use of a flying insect in a method of transferring a phage and/or immunogenic agent to an animal or plant, and to methods of treating or preventing disease in animals and plants using flying insects. The disclosure also relates to formulations of phages and/or immunogenic agents for use in treating and/or preventing disease in animals or plants.

### BACKGROUND

Flying insects such as flies are known to transmit bacterial, parasitic, and viral diseases to animal and plant species. Flies pick up and carry microorganisms from surfaces they come into contact with, including, inter alia, human and animal waste and decaying organic material. When flies subsequently make physical contact with animals or plants, they transmit the microorganisms that they are carrying to the animal and plant hosts, thus acting as mechanical vectors for microorganisms.

One example of a fly which is a common vector for bacterial diseases is Musca autumnalis, which is commonly referred to as the face fly and is a pest species for bovine and equine animals that is indigenous to Europe and Central Asia and found throughout Canada and most of the United States. Other Muscidae family members including the Australian bush fly (Musca vetustissima) and the buffalo fly (Haematobia exigus) are flies which may act as vectors for bacterial diseases in southern hemisphere regions such as Australia. In the case of face flies, in the spring, they emerge from their overwintering sites and females deposit eggs in fresh cow dung. Depending on air temperature, adult flies live for 20 to 50 days and from early autumn the insect burden on bovine and equine animals declines as surviving flies prepare to overwinter. Although face flies can migrate several kilometres in search of hosts, once a suitable habitat is found they tend to congregate around cattle, particularly near watering places.

Face flies, particularly females, feed on the blood, sweat, saliva, nasal mucous, dung, and tears of bovine and equine hosts. Feeding occurs only during daylight hours, meaning that the face flies leave the cattle each evening and there is a redistribution of the face flies every morning. Fly numbers of the order of 5 to 30 per cattle face are typically recorded in the daytime in summer months.

A disease for which face flies commonly act as a vector is infectious bovine keratoconjunctivitis (IBK), which is the most common eye disease in cattle, also known by the pseudonyms New Forest Eye and Pinkeye. Its principal aetiological agent is a Gram-negative rod-shaped bacterium, Moraxella bovis, although other microorganisms including Moraxella catarrhalis, Moraxella bovoculi, and Mycoplasma bovoculi infection have also been linked with a potential predisposing role in the pathogenesis of IBK.

Face flies are a common carrier of Moraxella bovis bacteria and are thus a common vector for IBK. In particular, surfaces of face flies including their mouthparts, bodies and limbs can become contaminated with Moraxella bovis bacteria that may remain viable for hours, while Moraxella bovis bacteria ingested by face flies may remain viable for up to 3 days in the fly alimentary tract, meaning that face flies carrying Moraxella bovis can initiate eye infections of cattle for several days by regurgitation of droplets from the foregut and midgut.

The economic impact of this disease is considerable and IBK is the most common condition affecting beef and dairy heifers, and the second most common disease of nursing calves greater than three weeks old.

Currently, the most commonly used treatments for IBK are antibiotic medications, which are delivered via systemic injection, subconjunctival injection, intrapalpebral injection, or topical application. Alternatively, it has been necessary to rely on preventative measures such as the use of ear tags containing insecticide. However, all of these treatment options are compromised by the pharmacokinetics of drug delivery to the ocular surface and the need for animal handling and restraint in order for treatment or preventative measures to be administered. Therefore, there remains a need for an effective method of reducing the incidence of bacterial infection in animals, and in particular for reducing the incidence of face fly induced IBK in cattle, which does not require animal handling and restraint.

Cattle are inclined to spend periods of the day rubbing themselves against fence posts, trees, or bushes, during which they seem to derive enjoyment and satisfaction. Observation of this behaviour has culminated in the development of commercially produced brushes and body buffers that can be deployed within fields and enclosures to facilitate this activity. Rubbing of the head, in particular, gives rise to temporary displacement of face flies from the face of a cow, causing the flies to find another resting place or remain airborne until their return to the face can be achieved. It would be advantageous if this natural inclination of the cattle could be employed in a strategy to mitigate fly induced IBK or other bacterial infections in cattle.

Other fly species including horn flies (Haematobia irritans) and stable flies (Stomoxys calcitrans) are major pests of cattle. Like the common house fly (Musca domestica) and blow flies (Calliphora vomitoria), these fly species are vectors for dissemination of bacteria, and horn flies in particular are implicated in the transfer of bacteria, including staphylococcal species, that can cause mastitis in cattle. Thus, displacement from cattle of these nuisance flies and elimination of their bacterial burden is advantageous in the context of cattle health and welfare.

Another host species which is susceptible to bacterial diseases carried by flying insects is the banana plant (part of the Musa family). Banana plants are susceptible to several bacterial diseases including ‘banana bacterial wilt’ caused by the bacterium Xanthomonas campestris, and ‘Moko disease’ caused by the bacterium Ralstonia solancearum. Parasitic flies, such as the oriental fruit fly (Bactrocera dorsalis) and wasps are active in banana plantations, and can thus act as a vector for carrying bacterial diseases to banana plants.

Similarly, the olive tree (Olea europaea) is susceptible to bacterial diseases from bacteria carried by the Olive fruit fly (Bactrocera oleae), including the bacteria Xylella fastidiosa and Pseudomonas savastanoi.

Bees, such as bumble bees (Bombus terrestris audax) can also carry bacterial infections to plant species, such as flowers. In particular, many plants are susceptible to bacterial blight caused by one of several strains of Pseudomonas syringae which have a very wide range of host species. These pathogens can be carried and disseminated by bees and other flying insects. For example, commercial bee hives are commonly used to enhance pollination of fruit-bearing plants such as kiwifruit (Actinidia deliciosa) as a means of increasing fruit yield. However, kiwifruit plants are highly susceptible to infection caused by Pseudomonas syringae pv. Actinidiae which can be carried by bees. Thus, it would be greatly advantageous to limit the bacterial burden of bees in commercial bee hives as well as to allow them to distribute phages with activity against this pathogen to plants as a means of disease prevention. Presently, agricultural strategies for mitigating bacterial infections in plants rely heavily on artificial chemicals being introduced into the soil or applied to the plant species, which disrupts the surrounding ecosystem. Thus, there also remains a need for an effective method of reducing the incidence of bacterial infection in plants.

### BRIEF DESCRIPTION OF THE FIGURES

FIG. 1 shows an elevated view of a fly of the Muscidae family feeding on an attractant consisting of a 50:50 mixture of glucose syrup and water.

FIG. 2a shows an elevated view of a fly of the Muscidae family feeding on an attractant consisting of icing sugar and a separate source of water.

FIG. 2b shows a side view of a fly of the Muscidae family feeding on an attractant consisting of icing sugar and a separate source of water.

FIG. 3 shows the growth of Moraxella bovis on agar plates after they were left over the course of a day in enclosures containing flies, bacteriophages with lytic activity against Moraxella bovis in sucrose solution or both.

FIG. 4 shows the growth of Salmonella enterica on agar plates after they were left over the course of a day in enclosures containing flies, bacteriophages with lytic activity against Salmonella enterica in sucrose solution or both.

FIG. 5 shows the growth of Salmonella enterica pathogens on agar plates after transfer of solution used to rinse an ex vivo bovine eye that had been left over the course of a day in an enclosure containing face flies, bacteriophages with lytic activity against Salmonella enterica in sucrose solution or both.

FIG. 6 shows the growth of Salmonella enterica pathogens on agar plates after transfer of solution used to rinse a freshly cut flower that had been left over the course of a day in an enclosure containing bees, bacteriophages with lytic activity against Salmonella enterica in sucrose solution or both.

FIG. 7 shows a transmission electron microscopy image of a bacteriophage with activity against Moraxella bovis.

FIG. 8 shows a transmission electron microscopy image of a bacteriophage with activity against Moraxella bovis.

FIG. 9 shows a transmission electron microscopy image of a bacteriophage with activity against Moraxella bovis.

FIG. 10 shows a transmission electron microscopy image of a bacteriophage with activity against Moraxella bovis.

FIGS. 11a and 11b show clusters which were produced following serial passage of the isolate which resulted in the phage of FIG. 10.

### SUMMARY

In a first aspect of the disclosure, there is provided a method of attracting a flying insect to a phage and/or immunogenic agent, comprising either (i) combining the phage and/or immunogenic agent with a flying insect attractant or (ii) placing the phage or immunogenic agent within the attraction radius of a flying insect attractant.

A second aspect of the disclosure relates to the use of a flying insect in a method of transferring a phage and/or immunogenic agent to a terrestrial animal or plant, wherein the flying insect acts as a carrier of the phage and/or immunogenic agent.

A third aspect of the disclosure relates to a method of treating or preventing disease in a terrestrial animal or plant comprising transferring a phage and/or immunogenic agent to the animal or plant using a flying insect, wherein the flying insect acts as a carrier of the phage and/or immunogenic agent.

In a fourth aspect of the disclosure, there is provided a composition for the treatment or prevention of a bacterial disease in a terrestrial plant or animal, comprising (i) a phage and/or an immunogenic agent and (ii) a flying insect attractant selected from one or more of faecal matter, pheromones, volatile agents, odorous materials, materials which can reflect or emit light within the visible electromagnetic spectrum, materials reflecting or emitting electromagnetic radiation outside the visible spectrum, a flying insect nutrient source, and combinations thereof. There is also provided an article comprising the composition of the disclosure.

In a fifth aspect of the disclosure, there is provided a kit for the treatment or prevention of a bacterial disease in a terrestrial plant or animal, comprising (i) a phage and/or an immunogenic agent and (ii) a flying insect attractant selected from one or more of faecal matter, pheromones, volatile agents, odorous materials, materials which can reflect or emit light within the visible electromagnetic spectrum, materials reflecting or emitting electromagnetic radiation outside the visible spectrum, a flying insect nutrient source, or resting places to which flying insects are innately attracted, such as hives in the case of bees, and combinations thereof.

In some embodiments, the present disclosure involves the use of a phage. The use of a phage solves the aforementioned problems because the phage can act against the bacteria naturally carried by the flying insect in order to reduce the flying insect's potential for infecting terrestrial animals and plants. Furthermore, the phage can treat or control bacteria already infecting target animals and plants and the phage can then be transferred to non-infected organisms by the flying insect. In other words, the flying insect which usually acts as a vector of the bacteria responsible for causing disease in the terrestrial animal or plant becomes the vector of a phage which can act against the bacteria responsible for causing the disease. This provides a targeted means for reducing the incidence of bacterial infection in terrestrial animals and plants, without requiring any direct human intervention including handling, manual manipulation or restraint.

In some embodiments, the present disclosure involves the use of an immunogenic agent. The present disclosure additionally provides a means of delivery of an immunogenic agent for provocation of an immunogenic response without need of direct human intervention. This is achieved by combining the immunogenic agent with a flying insect attractant, or placing the immunogenic agent within the attraction radius of a flying insect attractant, such that transfer of the immunogenic agent to the flying insect is achieved. Subsequent transfer of immunogenic agent to a target animal or plant provides a targeted means for provoking a protective reaction in terrestrial animals and plants, without requiring any direct human intervention including handling, manual manipulation or restraint. It will be appreciated that immune responses via humoral or cell-mediated mechanisms do not occur in plants as they do in animals. Nonetheless, it is widely recognised that plants have an immune system and are capable of eliciting a protective reaction in response to pathogen molecules.

In some embodiments, the phage and/or immunogenic agent is combined with a flying insect attractant which comprises a nutrient source on which a flying insect is inclined to feed, thereby encouraging the ingestion of the phage or immunogenic agent. The phage or immunogenic agent is then transferred to the target organism during regurgitation of droplets of flying insect alimentary tract contents which occurs as a normal feature of flying insect feeding activity.

### Definitions

The term “flying insect” as used herein refers to any insect having the ability to fly. Thus, the term “flying insect” includes all insects having one or more pairs of wings.

The noun “fly” as used herein refers to any flying insect characterised by the use of only one pair of wings for flight. This includes, but is not limited to, dipteran species.

The term “Diptera” as used herein refers to insects of the order Diptera and the term “dipteran” refers to a single insect of the order Diptera.

The term “face fly” as used herein refers to the species Musca autumnalis.

The term “Australian bush fly” as used herein refers to the species Musca vetustissima.

The term “buffalo fly” as used herein refers to the species Haematobia exigus.

The term “oriental fruit fly” as used herein refers to the species Bactrocera dorsalis.

The term “olive fruit fly” as used herein refers to the species Bactrocera oleae.

The term “blow fly” as used herein refers to any insect in the species Calliphora vomitoria or Lucilia sericata.

The term “bumble bee” as used herein refers to any insect in the species Bombus terrestris or Bombus terrestris audax.

The term “hover fly” as used herein refers to any insect in the family Syrphidae.

The term “horn fly” as used herein refers to the species Haematobia irritans.

The term “stable fly” as used herein refers to the species Stomoxys calcitrant.

The term “immunogenic agent” as used herein refers to any agent capable of providing immune response in a target terrestrial organism.

The term “attractant” as used herein refers to any substance which attracts a target animal. Thus, for example, a “flying insect attractant” refers to any substance which attracts a flying insect. It will be appreciated that an attractant may be a substance other than a nutrient source, it may be a substance that comprises or contains a nutrient source, or it may be a place to which an insect is innately attracted, such as a hive.

A “terrestrial animal”, as referred to herein, is an animal that lives predominantly or entirely on land. A “terrestrial plant”, as referred to herein, is a plant that grows on, in, or from land.

The term “comprising” as used herein means that at least all of the listed elements must be present, but other elements that are not mentioned may also be present.

In the context of the present disclosure, “attraction radius” means the area within which a target animal is attracted to the attractant. For example, for an insect attractant which involves the use of smell to attract the insect, the attraction radius is the area within which the insect can smell the attractant. It will be appreciated that the attraction radius for a particular attractant and target animal can be determined by routine experimentation. For example, attraction radius can be determined by placing the attractant in a test chamber under normal atmospheric conditions and positioning the target animal at increasing distances from the attractant to determine the maximum distance from which the target animal is attracted to the attractant.

The term “combining” as used herein to refer to combinations of X and Y covers the mixing of X and Y, as well as bringing X into contact with Y. In some embodiments, the term “combining” means mixing. In some embodiments, the term “combining” means contacting the first component with the second component.

The terms “IBK”, “New Forest Eye” and “Pinkeye” as used herein all refer to the disease infectious bovine keratoconjunctivitis.

The term “virus” according to the present disclosure includes double-stranded or single-stranded RNA or DNA viruses, which infect cells of bacteria, plants and/or animals.

The terms “phage” and “bacteriophage” are used interchangeably herein to refer to bacteriophages, which are viruses that infect specific bacteria.

The term “substrate” according to the present disclosure is understood to mean any solid phase material to which a virus, such as a bacteriophage, may be immobilised.

As used herein, the term “about,” as applied to one or more values of interest, refers to a value that is similar to a stated reference value. In some embodiments, the term “about” refers to a range of values that fall within 25% of the reference value in either direction (greater than or less than the reference value). In some embodiments, the term “about” refers to values that fall 25 within 20%, 19%, 18%, 17%, 16%, 15%, 14%, 13%, 12%, 11%, 10%, 9%, 8%, 7%, 6%, 5%, 4%, 3%, 2%, 1%, or less of the stated reference value (except where such number would exceed 100% of a possible value).

### DETAILED DESCRIPTION

The present disclosure relates to the use of a flying insect in a method of transferring a phage and/or an immunogenic agent to a terrestrial animal or plant, wherein the flying insect acts as a carrier of the phage and/or immunogenic agent.

In addition, the disclosure relates to a method of treating or preventing disease in a terrestrial animal or plant comprising transferring a phage and/or immunogenic agent to the animal or plant using a flying insect, wherein the flying insect acts as a carrier of the phage and/or immunogenic agent. In some embodiments, the method of treating or preventing disease in a terrestrial animal or plant comprises transferring the phage and/or immunogenic agent directly to the site of infection, such as bacterial infection, on the terrestrial animal or plant.

The present disclosure also relates to a method of transferring a phage and/or an immunogenic agent to a flying insect, comprising either (i) combining the phage and/or immunogenic agent with a flying insect attractant, or (ii) placing the phage and/or immunogenic agent within the attraction radius of a flying insect attractant.

In some embodiments of the present disclosure the flying insect attractant is a flying insect attractant selected from faecal matter, pheromones, volatile agents, odorous materials, materials which can reflect or emit light within the visible electromagnetic spectrum, materials reflecting or emitting electromagnetic radiation outside the visible spectrum, resting places to which flying insects are innately attracted, a nutrient source or combinations thereof.

The present disclosure further relates to a composition for the treatment or prevention of a bacterial disease in a terrestrial plant or animal, comprising (i) a phage and/or an immunogenic agent and (ii) a flying insect attractant selected from faecal matter, pheromones, volatile agents, odorous materials, materials which can reflect or emit light within the visible electromagnetic spectrum, materials reflecting or emitting electromagnetic radiation outside the visible spectrum, a flying insect nutrient source or combinations thereof.

In addition, the disclosure relates to a kit for the treatment or prevention of a bacterial disease in a terrestrial plant or animal, comprising (i) a phage and/or an immunogenic agent and (ii) a flying insect attractant selected from faecal matter, pheromones, volatile agents, odorous materials, materials which can reflect or emit light within the visible electromagnetic spectrum, materials reflecting or emitting electromagnetic radiation outside the visible spectrum, resting places to which flying insects are innately attracted, a flying insect nutrient source or combinations thereof.

In the context of the present disclosure, “prevention” or “preventing” a bacterial disease encompasses any manner in which phages or immunogenic agents which are transferred to a flying insect may act against bacteria, whether the bacteria are carried by the insect itself or located on a terrestrial animal or plant. For example, the prevention of bacterial disease can occur by the phage or immunogenic agent acting against bacteria which are present on the flying insect, thus reducing the bacterial load of the flying insect and preventing bacterial disease from being spread by the insect. Alternatively, the prevention of bacterial disease can occur when the flying insect contacts a terrestrial animal or plant, thus depositing phage or immunogenic agent on the terrestrial animal or plant and preventing bacterial infection occurring on the animal or plant.

In some embodiments, the phage and/or immunogenic agent is an antimicrobial agent.

In other embodiments, the phage and/or immunogenic agent comprises a bacteriophage. In some embodiments, the bacteriophage is an engineered bacteriophage, i.e., a bacteriophage which has been prepared for the disclosed methods, compositions, kits and uses.

Bacteriophages are viruses which target specific bacteria and, upon infection of their host bacterium, can multiply either by a lytic or lysogenic pathway. Lytic bacteriophages work by infecting the bacterium and using it to replicate and produce more of the virus. Eventually, after sufficient virus replication, lysis of the bacterial cells releases a large number of new bacteriophages, which can themselves infect neighbouring bacteria. Lysogenic bacteriophages work by integrating their DNA into bacterial chromosomes and replicating alongside the host chromosome to produce new integrated viral DNA copies. Advantageously, bacteriophages are specific to the bacteria that they target, meaning that they can be used as antibacterial agents in a host without causing damage to the other bacteria in the host, unlike antibiotics which are less specific.

It will be appreciated that bacteriophages with activity against a particular bacteria can be isolated by the skilled person. For example, bacteriophages can be isolated by first culturing the relevant bacteria and plating it on agar to produce a growing lawn of bacteria. Then, liquid can be harvested from a suitable source of bacteriophages including but not limited to animal tissue and secretions, faecal matter, plant matter, soil, or water samples. For example, bovine rumen fluid, nasal and lacrimal secretions can be harvested to isolate bacteriophages with activity against Moraxella bovis, which is a bacteria found in bovine animals, and liquid can be harvested from porcine faecal matter to isolate bacteriophages with activity against Salmonella enterica, which is a bacteria found in porcine animals. The lawn of bacteria on agar can then be exposed to aliquots of the harvested liquid and, for samples where bacterial lysis is observed, bacteriophage lysate can be concentrated from them.

In some embodiments, the bacteriophage is stabilised. For example, it is stabilised as described in EP 1496919 B1, by immobilisation to a substrate. This is advantageous because it ensures that the phage retains its antibacterial activity while also improving its stability relative to a free phage.

In some embodiments, as described in EP 1496919 B1, the bacteriophage is immobilised to the substrate via covalent bonds formed between the substrate and the phage. In some embodiments, it is immobilised via its head, leaving the tail free. This ensures that the bacteriophage retains its activity, because the tail of the bacteriophage is involved in the phage's ability to recognise and infect specific bacteria.

In some embodiments, the flying insect is a dipteran. In some embodiments, the dipteran is a face fly, Australian bush fly, buffalo fly, horn fly, or stable fly. This is advantageous because these flies are a pest species for bovine and equine animals. Thus, the use of these flies to carry a phage and/or immunogenic agent provides an effective method for targeting bovine and equine species with these agents, because the flies will transfer the phage or immunogenic agent to the bovine and equine species. In some embodiments, the dipteran is a face fly.

In some embodiments, the phage and/or immunogenic agent has activity against a bacterium associated with disease in a terrestrial animal or plant. In some embodiments, the phage and/or immunogenic agent has activity against a bacterium associated with disease in a terrestrial animal. In some embodiments, the terrestrial animal or plant is a terrestrial animal. In some embodiments, it is a bovine or equine species, for example, a cow or calf. In some embodiments, the phage and/or immunogenic agent has activity against a pathogen implicated in the aetiology of infectious bovine keratoconjunctivitis. In these embodiments, the phage and/or immunogenic agent may have activity against one or more of Moraxella bovis, Moraxella catarrhalis, Moraxella bovoculi, and Mycoplasma spp. It will be appreciated that phages are capable of direct activity against bacteria whereas immunogenic agents exert their effect indirectly via provocation of an immune response against bacteria. Nonetheless, both can be said to have activity against bacterial species and this will be understood by those skilled in this field. Thus, the present methods, kits, and uses offer an efficient way for a phage or immunogenic agent to be delivered to animals to counteract disease-causing pathogens by natural processes, without requiring direct human intervention.

In some embodiments, the phage and/or immunogenic agent has activity against a bacterium associated with disease in a terrestrial plant. In some embodiments, the phage and/or immunogenic agent has activity against a pathogen implicated in the aetiology of banana bacterial wilt. In these embodiments, the phage and/or immunogenic agent may have activity against Xanthomonas campestris. In other embodiments, the phage and/or immunogenic agent has activity against a pathogen implicated in the aetiology of Moko disease. In these embodiments, the phage and/or immunogenic agent may have activity against Ralstonia solanacearum. In some embodiments, the flying insect is a wasp. In some embodiments, the flying insect is an oriental fruit fly. In other embodiments, the phage and/or immunogenic agent has activity against Xylella fastidiosa, Pseudomonas syringae, or Pseudomonas savastanoi. In some embodiments, the flying insect is an olive fruit fly. In a further embodiment the insect is a bee, for example a bumble bee. Thus, the present methods, kits, and uses offer an efficient way for a phage and/or an immunogenic agent to be delivered to plants to counteract disease-causing pathogens by natural processes, by harnessing the natural movements of insect species for plants, without requiring direct human intervention.

Additionally, the present disclosure relates to a method of transferring a phage and/or an immunogenic agent to a flying insect, comprising either combining the immunogenic agent with a flying insect attractant or placing the phage and/or immunogenic agent within the attraction radius of a flying insect attractant.

In some embodiments, the attractant comprises one or more of faecal matter, pheromones, volatile agents, odorous materials, materials which can reflect or emit light within the visible electromagnetic spectrum, materials reflecting or emitting electromagnetic radiation outside the visible spectrum, or resting places to which flying insects are innately attracted. Resting places to which flying insects are innately attracted include but are not limited to rubbing brushes, hives, and the surfaces of terrestrial plants and animals. For example, flies are innately attracted to the faces of cattle and bees are innately attracted to flowers. It will be appreciated that a nutrient source under appropriate circumstances may also act as an attractant. Thus, an attractant may comprise a combination of two substances: one which is a nutrient source and one which is not a nutrient source.

In some embodiments, the composition or kit comprises a flying insect attractant which comprises a flying insect nutrient source, or the method comprises combining the phage and/or immunogenic agent with a flying insect nutrient source. The nutrient source may be a sugar, for example glucose or dry icing sugar. In some embodiments, the combination of phage and/or immunogenic agent and nutrient source is presented in liquid form. In some embodiments, the combination of phage and/or immunogenic agent and nutrient source is presented in solid form.

The use of a flying insect attractant comprising a nutrient source is advantageous because it promotes the likelihood of ingestion of the phage and/or immunogenic agent by the insect when it feeds on the nutrient source. This is advantageous because it will ensure that the phage and/or immunogenic agent is transferred to the flying insect's alimentary tract. This is particularly advantageous when the flying insect is a fly, because the regurgitation of droplets of gut contents is a normal feature of fly feeding activity. As a result, when the fly moves from feeding on the nutrient source to feeding on an animal or plant host, there is a particularly high transmission of the phage and/or immunogenic agent to the animal or plant host.

In some embodiments, the phage and/or immunogenic agent is combined with a flying insect attractant which comprises a nutrient source on which the flying insect is inclined to feed, thereby encouraging ingestion of the agent by the flying insect.

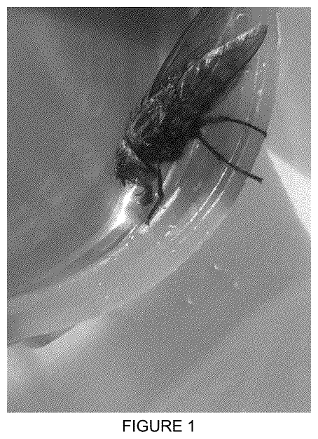

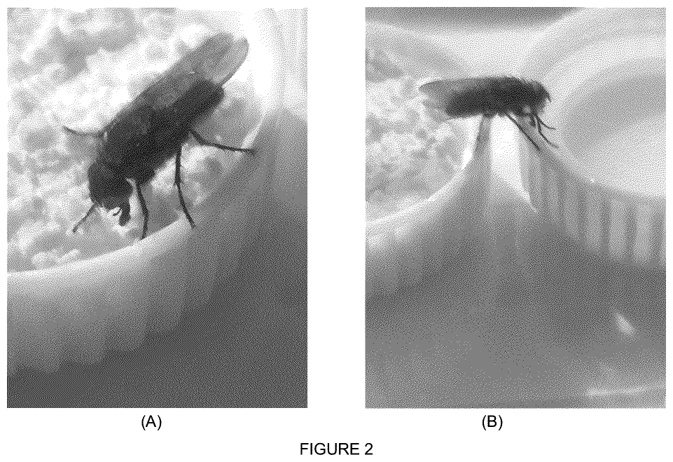

FIG. 1 illustrates an exemplary liquid nutrient source for flies which consists of a 50:50 mixture of glucose syrup and water. FIGS. 2a and 2b illustrate an exemplary solid nutrient source for flies, consisting of dry icing sugar. These figures show a fly feeding on each of the nutrient sources, demonstrating that when a phage and/or immunogenic agent that is useful for the treatment or control of a bacterium that causes disease in terrestrial organisms is combined with or within the attraction radius of the nutrient source, it will also be picked up by the fly by ingestion or inhalation or contamination of the extremities.

In some embodiments, the attractant comprises faecal material. The use of faecal material as an attractant is advantageous because insects frequently come into contact with faecal material throughout their lifecycle. In particular, for species of insect which lay eggs in dung, including the face fly, contact is made with faecal matter during each laying cycle. Thus, when faecal material is used as the attractant, this provides an efficient method for ensuring that the immunogenic agent is picked up by the dipteran, and thus will be subsequently transferred to a plant or animal host when the dipteran moves. In some embodiments, the faecal material comprises equine faecal material.

In some embodiments, the faecal material comprises bovine faecal material. This is advantageous for use in combination with phages and/or immunogenic agents having activity against bacteria that cause infections in bovine animals, such as cows. In addition, this is advantageous for insects that lay eggs in bovine faecal material. Taking the example of face flies, given that female face flies lay eggs in cattle dung, they are extremely likely to come into contact with phages and/or immunogenic agents if they are positioned in or on cattle dung. In addition, female face flies have a particularly high feeding rate on the blood, sweat, saliva, nasal mucous, dung, and tears of bovine and equine hosts. Therefore, a method involving bovine faecal material is likely to cause efficient transfer of immunogenic agents by face flies to bovine hosts.

In some embodiments, the phage and/or immunogenic agent is combined with the flying insect attractant. This is advantageous, for example, in the case of the attractant including a flying insect nutrient source, because it ensures that when the flying insect makes contact with or consumes some of the nutrient source it will also consume some of the phage and/or immunogenic agent.

In other embodiments, the phage and/or immunogenic agent is placed within the attraction radius of the attractant. This is advantageous in contexts where the phage and/or immunogenic agent cannot be combined with the attractant, because its location within the attraction radius of the attractant ensures that when the flying insect moves toward the attractant, the phage and/or immunogenic agent will be transferred to the flying insect.

It will be appreciated that the attraction radius will vary depending on the type, concentration, and potency of the attractant, as well as the identity of the target animal or plant. The attraction radius for a particular combination of an attractant and a target animal or plant is determined by routine experimentation. For example, attraction radius can be determined by placing the attractant in a test chamber under normal atmospheric conditions and positioning the target animal at increasing distances from the attractant to determine the maximum distance from which the target animal is attracted to the attractant. One example of a study determining an attraction radius for house flies using a poultry farm as an attractant is found in Nazni W A et. al, Trop Biomed. 2005 June; 22(1):53-61.

In some embodiments, the attraction radius may be within about one kilometer of the attractant. In some embodiments, the attraction radius may be within about five hundred metres of the attractant. In some embodiments, the attraction radius may be within about one hundred metres of the attractant. In some embodiments, the attraction radius may be within about ten metres of the attractant. In some embodiments, the attraction radius may be within about five metres of the attractant. In some embodiments, the attraction radius may be within about one metre of the attractant.

It will be appreciated that the optimal concentration of phage or immunogenic agent combined with attractant can be determined using challenge studies in which different concentrations are investigated. In some embodiments, the concentration of phage may be >10² pfu/ml, >10³ pfu/ml, >10⁴ pfu/ml, or >10⁶ pfu/ml. In some embodiments, the concentration of phage is about 10⁶ pfu/ml.

In some embodiments, the compositions or kits comprise two or more phages and/or immunogenic agents, or the use or method involves the use of two or more phages and/or immunogenic agents. In some embodiments, the composition includes a combination of two or more phages. The use of combinations of phages and/or immunogenic agents is advantageous because bacteria have evolved numerous defence mechanisms against viruses and are known to develop resistance. It is therefore desirable for preparations to comprise a cocktail of two or more active agents to minimise the impact of such resistance.

In some embodiments, the composition of the disclosure may be applied to a plastic substrate, which in turn may be attached to, or placed within an attraction radius of, a rubbing post for animals such as cattle or equine species. Alternatively, the plastic substrate may be in the form of a liquid or liquid glass. When it is a liquid formulation, it may be coated, absorbed or dispersed in other materials such as faecal material. In some embodiments, the composition of the disclosure may be placed inside a housing which protects the composition from external conditions, such as meteorological conditions.

For example, the plastic substrate may be attached to, or placed within an attraction radius of, a post, tree or other natural or artificial structure that animals such as cattle or equine species may come into contact with, for example rub against, or in the environment of. The structure may be a brush or body buffer.

This is advantageous in the case of cattle, where face flies act as a vector of disease. In particular, given that face flies are displaced from the faces of the cattle when the cattle rub their heads against posts, trees, or other structures such as brushes or body buffers, the flies either remain airborne or seek an alternative resting place until they can return to the faces of the cattle. Thus, the deployment of phages or immunogenic agents with activity against Moraxella bovis in combination with a fly attractant and positioned in conjunction with or in the vicinity of a rubbing station will promote their uptake by the flies. For example, a face rubbing brush with an integrated resting place for flies that contains a reservoir of phages/immunogenic agents, with or without fly attractant, will prompt the flies to rest upon the reservoir of phages/immunogenic agents when they are displaced from the faces of the cattle. The flies will then pick up the phages/immunogenic agents and transfer them to the faces of the cattle when they return, resulting in the treatment or prevention of bacterial infections such as IBK. This strategy is beneficial because it harnesses the natural behaviour of the cattle and face flies and requires no direct human intervention.

Alternatively, application of phages or immunogenic agents, with or without flying insect attractant, directly to the face rubbing brush would allow transfer of phages/immunogenic agents to the faces of cattle without direct human interaction. A further method of transferring phages/immunogenic agents to the faces of cattle without direct human interaction involves the addition of the phages/immunogenic agents, with or without flying insect attractant, to the rim of a vessel of suitable dimensions that contains a food source for cattle. Foraging and prehension of food from such a vessel would culminate in transfer of phages/immunogenic agents to the faces of cattle as their muzzles make contact with the vessel's rim. In both these cases, a flying insect can then transfer the phage/immunogenic agent from the face of the animal to the area of the animal to be treated, for example, the eye. This is particularly advantageous when a flying insect attractant is present, because this encourages flying insects to pick up the phages from the faces of the cattle and transfer them to the site of infection.

In some embodiments, the composition of the disclosure may be incorporated into a wearable article for terrestrial animals, such as an ear tag for a bovine or equine animal. Alternatively, the composition of the disclosure could be directly or indirectly adhered to the animal's integument. In these embodiments, the composition will be carried by the terrestrial animals if they are nomadic, rather than being fixed in one place. This is advantageous because the composition will move with the animals and continue to attract flying insects to the animals even if the animals move. An ear tag is particularly advantageous because the composition is located in the vicinity of the face of the animals, thus allowing easy access to flying insects which are attracted to the face of the animals, such as face flies.

In all of the above-recited embodiments, the final form of the composition will be such that it enables the flying insect to feed upon and ingest the phage and/or immunogenic agent.

### Examples

Experiments were performed to investigate the ability of flying insects to carry bacteriophages from one source to a remotely located site of infection. During these proof of principle experiments reproducibility and utility of the concept were demonstrated by employment of three different flying insect vectors and two different phage/pathogen systems. More specifically, the experiments were conducted with face flies (Musca autumnalis), blow flies (Calliphora vomitora), and bumble bees (Bombus terrestis audax) in combination with either bacteriophages with specificity for Moraxella bovis or bacteriophages with specificity for Salmonella enterca. In the case of the trials involving Salmonella entenca, bacteriophages were used that had been isolated previously against Salmonella enterica serovar Typhimurium SL1344. These Salmonella enterica phages were isolated by exposing growing lawns of Salmonella enterica on agar to aliquots of liquid harvested from porcine faecal matter than had been suspended in sterile aqueous buffer and subsequent observation of plaques in the bacterial lawns after a period of incubation at 37° C.

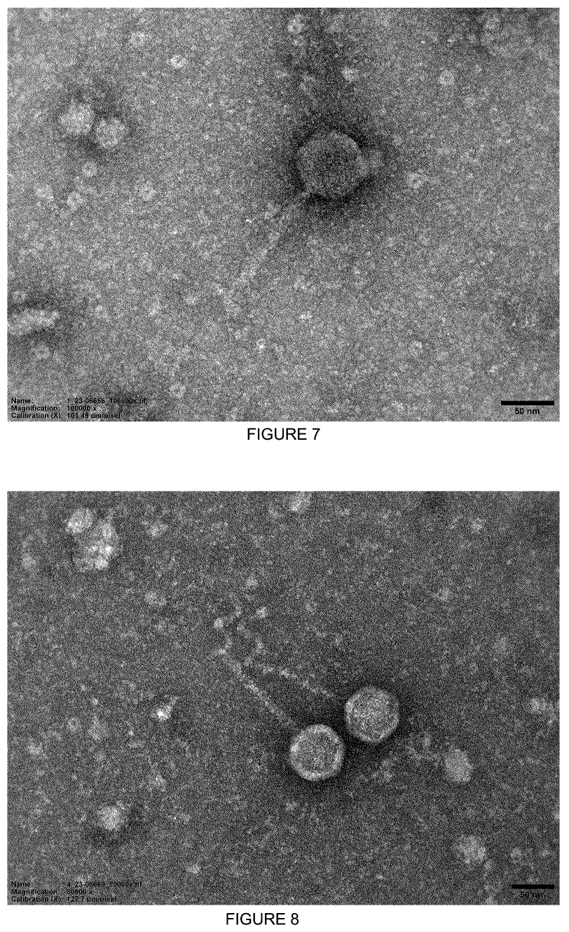

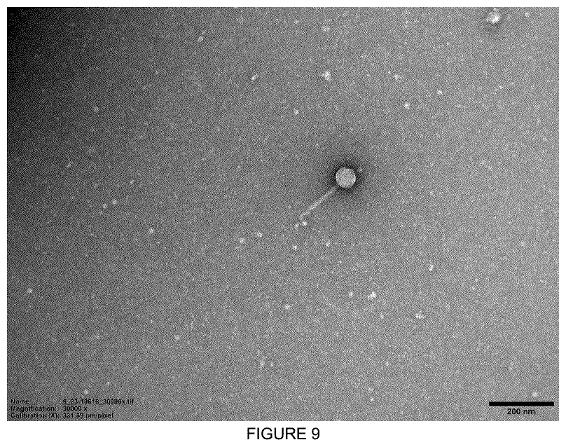

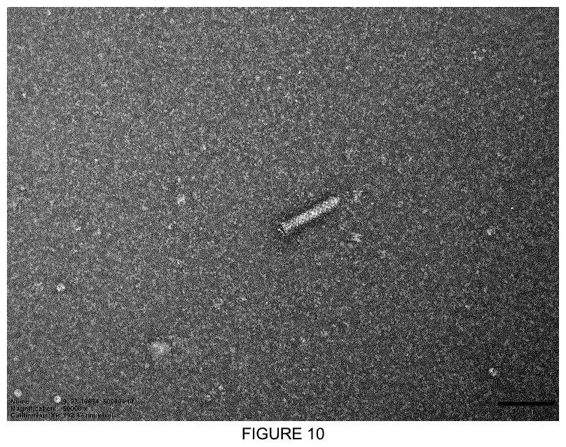

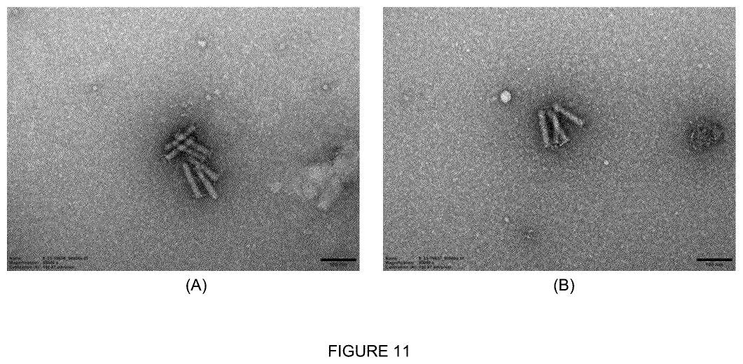

In the case of Moraxella bovis, bacteriophages were isolated as follows. Moraxella bovis was purchased from the National Collection of Type Cultures (NCTC 9426) and cultured in Brain Heart Infusion (BHI) broth supplemented with 5% horse blood. Bacteria were plated into BHI-agar supplemented with 5% horse blood and the plates were incubated at 37° C. for 48 hours. Bacteriophages with lytic activity against Moraxella bovis were isolated from abattoir specimens of bovine rumen fluid and bovine nasal and lacrimal secretions, from which bacteriophage lysate had been concentrated. This was achieved by introduction of the lysate to the plates of Moraxella bovis in blood-agar and observation of bacterial lysis after a period of further incubation. Inclusion of triphenyl tetrazolium chloride in the blood-agar plates aided visualisation of bacterial lysis by bacteriophages. Plaques were harvested for enrichment in Moraxella bovis/BHI broth supplemented with 5% horse blood. Serial passage was performed for four separate cultures to achieve clonal populations of bacteriophages which were submitted for transmission electron microscopy (TEM). TEM images for four phages with activity against Moraxella bovis which were isolated are shown in FIGS. 7, 8, 9 and 10. FIGS. 11a and 11b show clusters which were produced following serial passage of the isolate which resulted in the phage of FIG. 10. It can be seen that all of the phages in the isolate had identical morphology, consistent with a clonal population after serial passages. Further, it can be seen that each of the isolated phages had varying morphologies. While the phage in FIG. 7 had typical phage morphology, the phage in FIG. 10 had significantly different morphology. The phages in FIGS. 8 and 9 had very similar morphologies to one another.

### Example 1

A model system was established for experimental investigation of the ability of flies to carry bacteriophages from one source to a remotely located site of infection. In this model the site of infection was represented by growths of Moraxella bovis on blood-agar or Salmonella enterica on agar and the flying insect used was the face fly (Musca autumnalis) or the blow fly (Calliphora vomitoria). The face flies used in the experiment were wide-caught specimens taken from the vicinity of cattle. In the case of the blow flies, these were 3 day old adult flies recently hatched from pupae that had been incubated at 23° C. illuminated under a 12:12 light:dark cycle. Bacteriophages with lytic activity against Moraxella bovis or Salmonella enterica were added to a solution of 20% sucrose solution which acted as a flying insect attractant. In the case of the Moraxella bovis experiment, a cocktail of the four bacteriophages shown in FIGS. 7-10 was used. The concentration of bacteriophages with lytic activity against Moraxella bovis was about 10⁶ pfu/ml and the concentration of bacteriophages with lytic activity against Salmonella enterica was about 101 pfu/ml.

An enclosure was set up which contained the bacteriophages in sucrose solution, 5 adult flies and the agar plate with a growth of pathogen placed remotely from the sucrose solution. Thus, within the enclosure, flies were able to feed on the sucrose solution, in doing so consuming bacteriophages, and travel to a site of infection on the remotely located agar plate where they could additionally feed.

In addition, other enclosures were set up as controls that contained:
1. Control 1: 20% sucrose solution without bacteriophages, but with flies and a remotely located agar plate with a growth of Moraxella bovis or Salmonella enterica.
2. Control 2: Bacteriophages in 20% sucrose solution and an agar plate with a remotely located growth of Moraxella bovis or Salmonella enterica, but no flies.

Table 1 summarises this experimental set up for each of the Moraxella bovis and Salmonella enterica pathogen experiments.

TABLE 1
Summary of the setup of Example 1

| | Bacteriophages | Flying insect | Remotely located |
|---|---|---|---|
| | in 20% sucrose | (5 adult | growth |
| | solution | flies) | of pathogen |
| Control 1 | Absent | Present | Present |
| Control 2 | Present | Absent | Present |
| Enclosure according | Present | Present | Present |
| to the disclosure | | | |

Each enclosure was left at room temperature over the course of a day. The agar plate from each enclosure was then removed and analysed to determine whether there had been any effect on pathogenic growth.

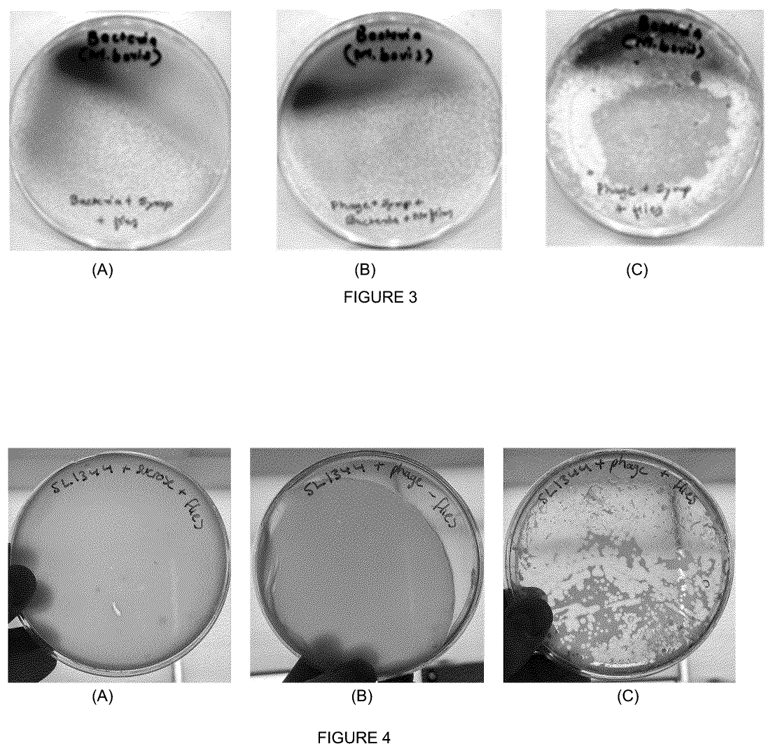

FIG. 3 shows each of the agar plates at the end of the experiment for Moraxella bovis. It can be seen that in the Control 1 (FIG. 3A) and Control 2 (FIG. 3B) iterations of the experiment the lawn of Moraxella bovis continued growing unimpeded. In contrast, in the iteration of the experiment in which both flies and bacteriophages in sucrose solution were present (FIG. 3C), areas were seen in which Moraxella bovis growth was depleted due to bacteriophage-mediated cell lysis. Visualisation of this lytic activity was enhanced by inclusion of triphenyl tetrazolium chloride.

FIG. 4 shows each of the agar plates at the end of the experiment for Salmonella enterica. It can be seen that In the Control 1 (FIG. 4A) and Control 2 (FIG. 4B) iterations of the experiment the lawn of Salmonella enterica continued growing unimpeded. In contrast, in the iteration of the experiment in which both flies and bacteriophages in sucrose solution were present (FIG. 4C), areas were seen in which Salmonella enterica growth was depleted due to bacteriophage-mediated cell lysis. Visualisation of this lytic activity was enhanced by inclusion of triphenyl tetrazolium chloride.

The results of Example 1 confirm that flying insects are capable of acting as a vector to transfer bacteriophages from one location to another and thus that, where bacterial infection exists, they can be a means of ameliorating infection at that second location.

### Example 2

A model system was established for experimental investigation of the ability of flying insects to carry bacteriophages from one source to a remotely located bovine eye. In this model, the flying insect used was the face fly (Musca autumnalis). The face flies used in the experiment were wide-caught which were maintained on 20% sucrose solution prior to the experiment under ambient conditions with a 12:12 light:dark lighting pattern. The bovine eye was an abattoir sourced ex vivo bovine eye. Bacteriophages with lytic activity against Salmonella enterica were added to a solution of 20% sucrose solution which acted as a flying insect attractant. The concentration of bacteriophages with lytic activity against Salmonella enterica was about 10¹¹ pfu/ml.

An enclosure was set up which contained the bacteriophages in sucrose solution, 5 adult flies and the bovine eye placed remotely from the sucrose solution. Thus, within the enclosure, flies were able to feed on the sucrose solution, in doing so consuming bacteriophages, and travel to the bovine eye.

In addition, other enclosures were set up as controls that contained:
1. Control 1: 20% sucrose solution without bacteriophages, but with flies and a remotely located ex vivo bovine eye;
2. Control 2: Bacteriophages in 20% sucrose solution, a remotely located ex vivo bovine eye, but no flies.

Table 2 summarises this experimental set up.

TABLE 2
Summary of the setup of Example 2

| | Bacteriophages | Vector (5 | Remotely |
|---|---|---|---|
| Experimental | in 20% sucrose | adult face | located ex vivo |
| conditions | solution | flies) | bovine eye |
| Control 1 | Absent | Present | Present |
| Control 2 | Present | Absent | Present |
| Enclosure according | Present | Present | Present |
| to the disclosure | | | |

Each enclosure was left for one day at room temperature. The bovine eye from each enclosure was then removed and the surface of the eye was rinsed with 1 ml of sterile PBS solution of which 100 μl were transferred to a growing lawn of Salmonella enterica on agar for overnight incubation at 37° C. The agar plate from each eye was then analysed to determine whether there had been any effect on pathogenic growth.

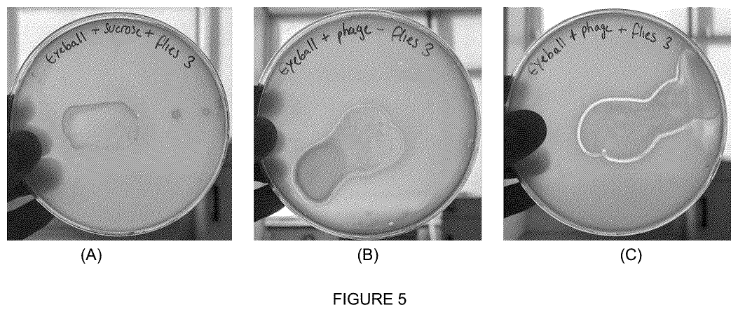

FIG. 5 shows each of the agar plates at the end of the experiment. In the Control 1 (FIG. 5A) and Control 2 (FIG. 5B) agar plates, the addition of the PBS effluent from the ex vivo bovine eye caused disturbance of the lawn of pathogens but areas of lysis of bacteria were not detected. In the iteration in which all variables were present (FIG. 5C), the addition of PBS effluent from the ex vivo eye caused disturbance of the lawn of pathogens and additionally lytic activity was seen where the liquid pooled.

The results of this experiment indicate that flies are capable of transferring bacteriophages from one location to a bovine eye where they have potential to limit or eradicate pathogens of a susceptible bacterial species.

### Example 3

A model system was established for experimental investigation of the ability of flying insects to carry bacteriophages from one source to a remotely located plant species. In this model, the flying insect used was the bumble bee (Bombus terrestris audax). The plant species was a freshly cut flower. Bacteriophages with lytic activity against Salmonella enterica were added to a solution of 20% sucrose solution which acted as a flying insect attractant. The concentration of bacteriophages with lytic activity against Salmonella enterica was about 101 pfu/ml.

An enclosure was set up which contained the bacteriophages in sucrose solution, 5 bumble bees and the freshly cut flower placed remotely from the sucrose solution. Thus, within the enclosure, bees were able to feed on the sucrose solution, in doing so consuming bacteriophages, and travel to the flower.

In addition, other enclosures were set up as controls that contained:
1. Control 1: Bees, a remotely located flower, but no bacteriophages in the 20% sucrose solution;
2. Control 2: Bacteriophages in 20% sucrose solution, a remotely located flower, but no bees.

Table 3 summarises this experimental set up.

TABLE 3
Summary of the setup of Example 3

| Experimental | Bacteriophages in | Vector | Remotely located |
|---|---|---|---|
| conditions | 20% sucrose solution | (5 bees) | freshly cut flower |
| Control 1 | Absent | Present | Present |
| Control 2 | Present | Absent | Present |
| Enclosure according | Present | Present | Present |
| to the disclosure | | | |

Each enclosure was left for one day at room temperature. The flower from each enclosure was then removed and rinsed with 1 ml of sterile PBS solution of which 100 μl were transferred to a growing lawn of Salmonella enterica on agar for overnight incubation at 37° C. The agar plate from each flower was then analysed to determine whether there had been any effect on pathogenic growth.

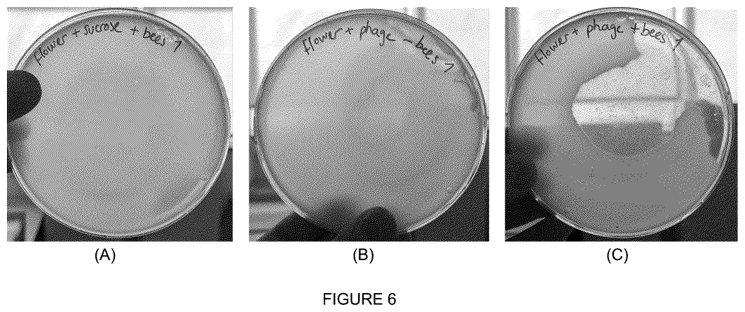

FIG. 6 shows each of the agar plates at the end of the experiment. In the Control 1 (FIG. 6A) and Control 2 (FIG. 6B) iterations of example 3, the addition of the PBS solution effluent from the flower did not give rise to any visible areas of lysis of bacteria. In contrast, for the iteration in which all variables were present (FIG. 6C), the addition to the growing lawn of Salmonella enterica of the PBS flower effluent gave rise to obvious lytic activity.

The results of this experiment indicate that insect vectors are capable of transferring bacteriophages from one location to plant species where they have potential to limit or eradicate pathogens of a susceptible bacterial species. Moreover, the results of this experiment reveal bees to be good vectors for dissemination of bacteriophages to flowering plants which has advantageous implications for crop protection at the time of plant pollination. Indeed, it would be reasonable to conclude that addition to a commercial bee hive of bacteriophages with activity against a particular pathogen would diminish the presence of that pathogen within the hive's bee population as well as on the vegetation visited by the bees.

### Example 4

A model system was established for experimental investigation of the ability of flying insects to carry bacteria from one source to a remote location.

The bacteria was Moraxella bovis which was cultured in BHI medium supplemented with 5% horse blood. A portion of this broth was added to sucrose to create a 20% solution containing bacteria. The flying insect used was the blow fly (Calliphora vomitoria).

An enclosure was set up which contained the bacteria in sucrose solution, 5 blow flies and a sterile blood-agar plate located remotely from the sucrose solution. Thus, within the enclosure, flies were able to feed on the sucrose solution, in doing so consuming bacteria, and travel to the sterile agar plate.

In addition, another enclosure was set up as a control, which contained the bacteria in sucrose solution and a sterile blood-agar plate located remotely from the sucrose solution, but no blow flies.

TABLE 4
Summary of the setup of Example 4

| Experimental | Bacteria in 20% | Vector | Sterile blood-agar |
|---|---|---|---|
| conditions | Sucrose solution | (5 flies) | plate |
| Control | Present | Absent | Present |
| Enclosure according | Present | Present | Present |
| to the disclosure | | | |

Each enclosure was left for one day at room temperature. Then, the blood-agar plates were retrieved and incubated at 37° C. The blood-agar plate retrieved from the enclosure that was free of flies failed to reveal bacterial growth. However, the blood-agar plate retrieved from the enclosure that had contained flies yielded visible colonies of bacteria.

This experiment confirms that flies are capable of transferring bacteria from one source to a remote location. It will be generally understood that bacteria are immunogenic and, indeed, vaccines can be developed from inactivated or killed bacterial or from bacterial components such as cell membrane proteins. Thus, it is implicit that flying insects are capable of carrying immunogenic substances from one location to another and it will be generally understood that supply of such substances in conjunction with a fly attractant together with a flying insect vector could be used to disseminate those immunogenic substances, including to animals and plants.

### Numbered Embodiments of the Disclosure

1. A composition for the treatment or prevention of a bacterial disease in a terrestrial plant or animal, comprising:
   (i) a phage and/or an immunogenic agent; and
   (ii) a flying insect attractant.
2. A method of attracting a flying insect to a phage and/or immunogenic agent, comprising either:
   (i) combining the phage and/or immunogenic agent with a flying insect attractant; or
   (ii) placing the phage and/or immunogenic agent within the attraction radius of a flying insect attractant.
3. The method of embodiment 2, wherein the phage and/or immunogenic agent is transferred to the flying insect when it moves toward or comes into contact with the attractant
4. A kit for the treatment or prevention of a bacterial disease in a terrestrial plant or animal, comprising:
   (i) a phage and/or immunogenic agent; and
   (ii) a flying insect attractant.
5. Use of a flying insect in a method of transferring a phage and/or an immunogenic agent to a terrestrial animal or plant, wherein the flying insect acts as a carrier of the phage and/or immunogenic agent.
6. The use of embodiment 5, wherein the phage and/or immunogenic agent has been transferred to the flying insect by the method of embodiment 2 or 3.
7. A method of treating or preventing disease in a terrestrial animal or plant comprising transferring a phage and/or immunogenic agent to the animal or plant using a flying insect, wherein the flying insect acts as a carrier of the phage and/or immunogenic agent.
8. The method of embodiment 7, wherein the phage and/or immunogenic agent has been transferred to the flying insect by the method of embodiment 2 or 3.
9. The composition of embodiment 1, the method of embodiment 2, 3 or 8, the kit of embodiment 4 or the use of embodiment 6, wherein the flying insect attractant comprises a flying insect nutrient source.
10. The composition, method, kit or use of embodiment 9, wherein the flying insect attractant comprises a flying insect nutrient source in combination with a second substance.
11. The composition of embodiment 1, 9 or 10, the method of embodiment 2, 3, 7, 8, 9 or 10, the kit of embodiment 4, 9 or 10, or the use of embodiment 5, 6, 9 or 10, wherein the phage and/or immunogenic agent has activity against a bacterium associated with disease in a terrestrial animal or plant.
12. The composition, or kit of any preceding embodiment, wherein the composition comprises two or more immunogenic agents and/or bacteriophages, preferably comprising two or more bacteriophages.
13. The method or use of any preceding embodiment, wherein two or more immunogenic agents and/or bacteriophages are used.
14. The composition or kit of any preceding embodiment, comprising a bacteriophage, or the method or use of any preceding embodiment wherein a bacteriophage is used.
15. The composition, method, kit or use of embodiment 14, wherein the bacteriophage is stabilised by immobilisation to a substrate, preferably wherein the bacteriophage is immobilised via its head.
16. The composition, method, kit or use of any preceding embodiment, wherein the immunogenic agent comprises a denatured bacterium antigen.
17. The composition, method, kit or use of any preceding embodiment, wherein the flying insect is a fly, preferably a dipteran, preferably wherein the flying insect is a face fly, Australian bush fly or buffalo fly, most preferably a face fly.
18. The composition, method, kit or use of any preceding embodiment, wherein the flying insect is a wasp, oriental fruit fly or olive fruit fly.
19. The composition, method, kit or use of any preceding embodiment, wherein the phage and/or immunogenic agent has activity against a bacterium associated with disease in a terrestrial animal, preferably wherein the animal is a bovine or equine animal, more preferably a bovine animal.
20. The composition, method, kit or use of any preceding embodiment, wherein the phage and/or immunogenic agent has activity against a bacterium associated with disease in a terrestrial plant, preferably wherein the plant is a banana plant or olive tree.
21. The composition, method, kit or use of any preceding embodiment, wherein the phage and/or immunogenic agent has activity against a pathogen implicated in the aetiology of infectious bovine keratoconjunctivitis.
22. The composition, method, kit or use of any preceding embodiment, wherein the phage and/or immunogenic agent has activity against Moraxella bovis.
23. The composition, method, kit or use of any preceding embodiment, wherein the phage and/or immunogenic agent has activity against Moraxella catarrhalis, Moraxella bovoculi, Mycoplasma spp, Xanthomonas campestris, Ralstonia solancearum, Xylella fastidiosa and/or Pseudomonas savastanoi.
24. The composition, method, kit or use of any preceding embodiment wherein a flying insect attractant is present, preferably wherein the attractant comprises one or more of faecal matter, pheromones, volatile agents, odorous materials, materials which can reflect or emit light within the visible electromagnetic spectrum, materials reflecting or emitting electromagnetic radiation outside the visible spectrum, or resting places to which flying insects are innately attracted.
25. The composition, method, kit or use of any preceding embodiment, wherein the attractant comprises one or more natural and/or synthetic substances, preferably one or more substances selected from pheromones, volatile agents and odorous materials, preferably a volatile agent.
26. The composition, method, kit or use of any preceding embodiment, wherein the attractant comprises a material reflecting or emitting light within the visible electromagnetic spectrum or a material reflecting or emitting electromagnetic radiation outside the visible spectrum.
27. The composition, method, kit or use of any preceding embodiment, wherein the flying insect attractant comprises a flying insect nutrient source, wherein the nutrient source is a sugar.
28. The composition, method, kit or use of any preceding embodiment, wherein the attractant and the phage and/or immunogenic agent are presented in solid form, or liquid form.
29. The composition, method, kit or use of any preceding embodiment, wherein the attractant comprises faecal matter, preferably bovine faecal matter.
30. The composition, method, kit or use of any preceding embodiment, wherein the phage and/or immunogenic agent is mixed with the attractant.
31. An article comprising the composition of any preceding embodiment.
32. The article of embodiment 31, wherein the article is wearable by terrestrial animals.
33. The article of embodiment 32, wherein the article is a bovine or equine ear tag.
34. A method wherein the composition of any preceding embodiment is applied to the integument of a terrestrial animal.

The present disclosure provides exemplary embodiments and is not intended to be limiting. It will be appreciated that various other modifications and variations of the disclosure are also possible.

1. A composition for the treatment or prevention of a bacterial disease in a terrestrial plant or animal, comprising:
(i) a phage and/or an immunogenic agent; and
(ii) a flying insect attractant selected from one or more of: faecal matter, a pheromone, a volatile agent, an odorous material, a material that can reflect or emit light within the visible electromagnetic spectrum, a material that can reflect or emit electromagnetic radiation outside the visible spectrum, a resting place to which flying insects are innately attracted, and a flying insect nutrient source.

2. The composition of claim 1, wherein the flying insect attractant is a flying insect nutrient source comprising a sugar.

3. The composition of claim 1, comprising two or more phages and/or immunogenic agents.

4. The composition of claim 1, wherein the phage is immobilized to a substrate.

5. The composition of claim 4, wherein the phage is immobilized to the substrate by a link between the head of the phage and the substrate.

6. The composition of claim 1, wherein the immunogenic agent comprises a bacterium antigen.

7. The composition of claim 1, wherein the flying insect attractant and the phage and/or immunogenic agent are present in solid form or liquid form; and/or wherein the flying insect attractant and the phage and/or immunogenic agent are present as a mixture.

8. The composition of claim 5, wherein the substrate is an article wearable by the terrestrial animal.

9. The composition of claim 8 wherein the wearable article is an ear tag.

10. A method of treating or preventing disease in a terrestrial animal or plant comprising transferring an effective amount of a phage and/or immunogenic agent to the terrestrial animal or plant using a flying insect, wherein the flying insect acts as a carrier of the phage and/or immunogenic agent.

11. The method of claim 10, wherein the phage and/or immunogenic agent is or has been transferred to the flying insect by:
(i) combining the phage and/or immunogenic agent with a flying insect attractant; or
(ii) placing the phage and/or immunogenic agent within the attraction radius of a flying insect attractant.

12. The method of claim 10, wherein the flying insect is a fly, a wasp, an oriental fruit fly, an olive fruit fly, a hover fly, or a bee.

13. The method of claim 12, wherein the fly is a face fly, an Australian bush fly, or a buffalo fly.

14. The method of claim 13, wherein the fly is a face fly.

15. The method of claim 10, wherein the phage and/or immunogenic agent has activity against:
a) a bacterium associated with a disease in a terrestrial animal and/or a terrestrial plant;
b) a pathogen implicated in the aetiology of infectious bovine keratoconjunctivitis; and/or
c) one or more of Moraxella bovis; Moraxella catarrhalis; Moraxella bovoculi; Mycoplasma spp; Xanthomonas campestris; Ralstonia solanacearum; Xylella fastidiosa; Pseudomonas savastanoi; and Pseudomonas syringae.

16. The method of claim 10, wherein the terrestrial animal is a bovine or equine animal.

17. The method of claim 10, wherein the terrestrial plant is a banana plant, an olive tree, or a kiwi fruit plant.

18. The method of claim 10, wherein the phage and/or immunogenic agent is transferred to the integument of the terrestrial animal.

19. A kit for the treatment or prevention of a bacterial disease in a terrestrial plant or animal, comprising:
(i) a phage and/or immunogenic agent; and
(ii) a flying insect attractant selected from one or more of: faecal matter, a pheromone, a volatile agent, an odorous material, a material that can reflect or emit light within the visible electromagnetic spectrum, a material that can reflect or emit electromagnetic radiation outside the visible spectrum, a resting place to which flying insects are innately attracted, and a flying insect nutrient source.

20. The kit of claim 19, wherein the flying insect attractant is bovine faecal matter.
